(19) United States
(12) Patent Application Publication
(10) Pub. No.: US 2003/0165468 A1
(43) Pub. Date: Sep. 4, 2003

(54) Regulated vectors for controlling DNA hypermutability in eukaryotic cells

(76) Inventors: Luigi Grasso, Bala Cynwyd, PA (US); Nicholas C. Nicolaides, Boothwyn, PA (US); Philip M. Sass, Audubon, PA (US)

Correspondence Address: WOODCOCK WASHBURN LLP, ONE LIBERTY PLACE, 46TH FLOOR, 1650 MARKET STREET, PHILADELPHIA, PA, 19103, US

(21) Appl. No.: 10/371,857

(22) Filed: Feb. 21, 2003

Related U.S. Application Data
(60) Provisional application No. 60/358,602, filed on Feb. 21, 2002.

(51) Int. Cl. A61K 48/00; C12N 1/18; C12N 5/04; C12N 5/06

(52) U.S. Cl. 424/93.2; 435/320.1; 435/325; 435/419; 435/348; 435/254.2

(57) ABSTRACT

The present invention features mammalian expression vectors that are useful for controlling DNA hypermutability in mammalian cell as well as the encoding polynucleotide sequences of vector sequences. In related aspects the invention features expression vectors and host cells comprising such polynucleotides. In other related aspects, the invention features transgenic cells expressing a mutator gene to enhance genome-wide mutagenesis, due to, for example, the presence of an exogenous mutator-encoding polynucleotide sequence. Further, the invention provides methods for using vector sequences that can remove the expression of such gene to restore DNA stability in a host cell.

### CROSS-REFERENCE TO RELATED APPLICATIONS

This application claims the benefit of U.S. Provisional Application No. 60/358,602, filed Feb. 21, 2002, the disclosure of which is hereby incorporated by reference in its entirety.

### FIELD OF THE INVENTION

The present invention relates to expression vectors containing nucleic acid sequences encoding for a mutator gene linked to a negative selection marker that can be used to positively and negatively regulate DNA hypermutability in eukaryotic cells.

### BACKGROUND OF THE INVENTION

The use of expressing mutator genes, such as but not limited to dominant negative inhibitors of mismatch repair, is a method for generating genetically altered cells to produce subtypes with desired phenotypes. The ability to restore genomic stability via suppressing the expressed mutator allele is critical for stabilizing the genetic integrity of the host's genome. While several methods for regulating gene expression from ectopic vectors exist, the ability to “completely” suppress expression of a mutator transgene is problematic, especially when complete suppression is required to ensure genetic stability. Here, we describe a eukaryotic expression vector system that is capable of producing robust expression levels of a given allele that can be regulated via negative selection to ensure complete suppression.

### SUMMARY OF THE INVENTION

It is the object of the present invention to teach the use of a vector system capable of complete suppression of a mutator transgene within eukaryotic cells.

The invention described herein is the development of a eukaryotic expression vector system for the use in generating genetically evolved cells in a controllable manner. This system entails a constitutively active promoter upstream of a cloning site containing a polylinker suitable for cloning a mutator gene of interest followed by an Internal Ribosome Entry Site and a second polylinker containing a negative selection marker and a polyadenylation site. Internal Ribosome Entry Sites (IRES) are regulatory elements that are found in a number of viruses and cellular RNAs (reviewed in McBratney et al. (1993) Current Opinion in Cell Biology 5:961). IRES are useful in enhancing translation of a second gene product in a linked eukaryotic expression cassette (Kaufman R. J. et al. (1991) Nucl. Acids Res. 19:4485). This system allows for the constitutive expression of a fusion transcript consisting of an effector mutator gene cDNA followed by a cDNA encoding for a negative selection marker that can be used to select for subclones within a population of cells to identify those that have naturally lost expression.

For the purposes of example, here we demonstrate the development and application of one such vector plasmid referred to as pIRES-PMS134-TK. The pIRES-PMS134-TK plasmid contains a dominant negative mismatch repair gene allele followed by an IRES signal and a negative selection marker derived from herpes simplex virus thymidine kinase (HSV-TK) gene. This vector when transfected in mammalian cells is capable of blocking the endogenous mismatch repair process, leading to subclones with altered phenotypes. Once a subclone with a desired phenotype is identified, the line is made stable through the treatment of ganciclovir (GCV), a prodrug that is converted into a toxic nucleoside analog in cells expressing the HSV-TK gene [Carrio M, Mazo A, Lopez-Iglesias C, Estivill X, Fillat C. (2001) “Retrovirus-mediated transfer of the herpes simplex virus thymidine kinase and connexin26 genes in pancreatic cells results in variable efficiency on the bystander killing: implications for gene therapy.” Int. J. Cancer 94:81-88]. Because the mutator cDNA and the HSV-TK are produced from the same transcript, clones that survive GCV treatment are naturally non-expressing the fusion transcript, and exhibit a mismatch repair proficient activity within the host cell.

The use of fusion transcripts containing a mutator gene such as those described by Wood et al., [Wood, R. D., Mitchell, M., Sgouros, J., and Lindahl, T. (2001) “Human DNA Repair” Genes Science 1284-1289] and a negative selection marker such as but not limited to the use of vectors containing HSV-tk for ganciclovir selection or the bacterial purine nucleoside phosphorylase gene for 6-methylpurine selection [Gadi V. K., Alexander S. D., Kudlow J. E., Allan P., Parker W. B., Sorscher E. J. (2000) “In vivo sensitization of ovarian tumors to chemotherapy by expression of E. coli purine nucleoside phosphorylase in a small fraction of cells” Gene Ther. 7:1738-1743] has advantages for recombinant methods employing mutator expression vectors to generate genetically diverse clones with de novo phenotypes, which in turn can be selected to identify a subset with restored genomic stability.

The vectors of the invention can be employed with the use of a dominant negative inhibitor of MMR in such those previously described [Nicolaides, N. C. et al. (1998) “A naturally occurring hPMS2 mutation can confer a dominant negative mutator phenotype” Mol. Cell. Biol. 18:1635-1641; U.S. Pat. No. 6,146,894 to Nicolaides et al.] as an example for developing new cell lines for discovery and product development. A PMS2 homolog may also be encoded refers to a polypeptide sequence having the consensus sequence of AVKE LVENSLDAGA TN (SEQ ID NO: 23). In some embodiments, the PMS2 homologs comprise the polypeptide sequence of LRPNAVKE LVENSLDAGA TNVDLKLKDY GVDLIEVSGN GCGVEEENFE (SEQ ID NO: 24). The PMS2 homologs comprise this structural feature and, while not wishing to be bound by any particular theory of operation, it is believed that this structural feature correlates with ATPase activity due to the high homology with known ATPases. The knowledge of this structural feature and correlated function and the representative number of examples provided herein, will allow one of ordinary skill in the art to readily identify which proteins may be used in the methods of the invention.

For example, in some embodiments of the invention, the PMS2 homologs may be truncated to lack a C-terminal domain believed to be involved with dimerization. While not wishing to be bound by any particular theory of operation, it is believed that some truncated PMS2 homologs may function by binding ATP as monomers, and mismatch repair activity is decreased due to insufficient heterodimerization between PMS2 and MLH1, however, the conserved N-terminus is intact. Thus, in some embodiments, the PMS2 homologs may further comprise a C-terminal truncation in which the fourth ATP binding motif is eliminated. In some embodiments, the PMS2 homologs comprise a truncation mutation such that the PMS2 homolog is incapable of dimerizing with MLH1. In other embodiments, the PMS2 homologs comprise a truncation from the E′ α-helix to the C-terminus (see FIG. 3 and Guarne et al. (2001) EMBO J. 20(19):5521-5531). In other embodiments, the PMS2 homologs comprise a truncation from the E α-helix to the C-terminus (see FIG. 3 and Guarne et al. (2001) EMBO J. 20(19):5521-5531). In other embodiments, the PMS2 homologs comprise a truncation from the F α-helix to the C-terminus (see FIG. 3 and Guarne et al. (2001) EMBO J. 20(19):5521-5531). In other embodiments, the PMS2 homologs comprise a truncation from the G α-helix to the C-terminus (see FIG. 3 and Guarne et al. (2001) EMBO J. 20(19):5521-5531). In other embodiments, the PMS2 homologs comprise a truncation from the H′ α-helix to the C-terminus (see FIG. 3 and Guarne et al. (2001) EMBO J. 20(19):5521-5531). In other embodiments, the PMS2 homologs comprise a truncation from the H α-helix to the C-terminus (see FIG. 3 and Guarne et al. (2001) EMBO J. 20(19):5521-5531). In other embodiments, the PMS2 homologs comprise a truncation from the I α-helix to the C-terminus (see FIG. 3 and Guarne et al. (2001) EMBO J. 20(19):5521-5531). Any PMS2 homolog that can serve to impart a dominant negative phenotype may be used with the vectors of the invention.

The invention described herein is directed to the creation of selectable expression vectors that can produce mutator proteins that can be suppressed upon negative selection to restore genomic stability.

The present invention describes the development of one such system. The advantages of the present invention are further described in the examples and figures described herein employing dominant negative alleles of MMR linked to the HSV-TK gene as a negative selection marker.

The invention also describes the development and composition of a specific vector that contains the dominant negative MMR gene cDNA PMS134 linked to the HSV-TK negative selection marker as shown in FIG. 1.

The invention also provides methods for generating pools and libraries of hypermutable somatic cell lines using methods known by those skilled in the art that can be negatively selected to identify subclones no longer expressing the mutator allele.

The invention also provides methods for generating hypermutable somatic cell lines using methods known by those skilled in the art that can be negatively selected to identify subclones no longer expressing the dominant negative allele, thus exhibiting a genetically stable host.

These and other objects of the invention are provided by one or more of the embodiments described below.

In one embodiment of the invention, a method for making a eukaryotic cell line genetically unstable, followed by selection of a new genotype and reintroduction of a genomic stability is provided. A polynucleotide encoding a dominant negative allele of a MMR gene is introduced into a target cell and the cell line is rendered MMR defective. Pools are generated and selected for subclones with novel phenotypes. Upon confirmation of desired subclones, subclones are expanded and negatively selected for subsets no longer expressing the dominant negative MMR gene allele.

In other embodiments, expression vectors comprising the fusion transcript are able to transform mammalian cells to generate high expression of mutator protein. Thus, another embodiment of the invention is an expression vector comprising a mutator gene fused to IRES sequence followed by a negative selection marker. In a preferred embodiment, the expression vector further comprises a eukaryotic promoter/enhancer driving the expression of a mutator protein of interest. In a most preferred embodiment, the expression vector consists of a fusion plasmid wherein a first gene encodes the gene of interest and a second gene encodes a negative selectable marker. Negative selectable markers include, but not limited to the herpes simplex virus thymidine kinase gene (HSV-TK) or derivatives thereof; other negative selectable markers are also suitable for use in the inventive expression vectors. The fusion expression vector may further comprise an IRES sequence between the two genes. Mammalian cells can be transformed with the expression vectors of the invention, and will produce recombinant mutator protein in a short period of time. Accordingly, another embodiment of the invention provides a mammalian host cell transformed with an expression vector of the invention. In a most preferred embodiment, the host cells are mammalian cells derived of rodent or human origin.

The invention also provides a method for obtaining a cell line with a new phenotype and a stable genome, comprising transforming a host cell with an inventive expression vector, culturing the transformed host cell under conditions promoting expression of the mutator protein, and selecting for new phenotypes. In a preferred application of this invention, transformed host cell lines are selected with two selection steps, the first to select for cells with a new phenotype, and the second step for negative selection of the marker gene to isolate subclones of the cell line exhibiting the desired phenotype that no longer express the mutator protein, thus having restored genomic stability. In a most preferred embodiment, the selection agent is ganciclovir, a prodrug that has been shown to cause toxicity to cells expressing the HSV-TK gene.

In some embodiments, cells may be cultured with a mutagen to increase the frequency of genetic mutations in the cells. The mutagen may be withdrawn upon identification and selection of cells displaying the desired altered phenotype.

These and other embodiments of the invention provide the art with methods that can regulate the genomic stability of a mammalian cell line using genes encoding for mutator proteins linked to a second gene that is useful for negatively selecting clones that no longer express the mutator gene allele.

### BRIEF DESCRIPTION OF THE DRAWINGS

FIG. 1 shows a schematic diagram of a regulated mutator expression plasmid.

FIG. 2 shows the expression of the mutator plasmid before and after negative selection.

FIGS. 3A and B show the structure of the N-terminal fragment of PMS2 (orthagonal views) and

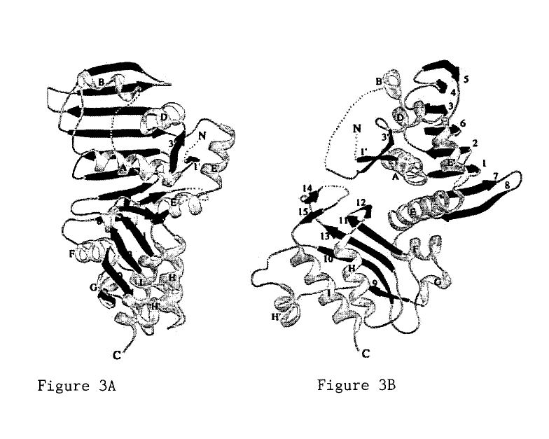

FIG. 3C shows a sequence alignment of hPMS2, hMLH1 and MutL N-terminal fragments and structural features corresponding to FIGS. 3A and B (from Guarne et al. (2001) EMBO J. 20(19):5521-5531, FIGS. 2A-C).

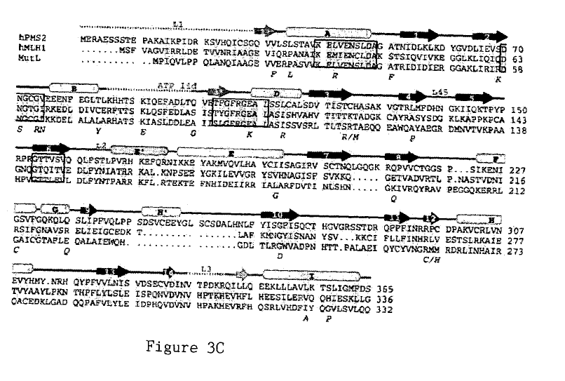

### DETAILED DESCRIPTION OF THE INVENTION

The invention provides novel expression vectors that can regulate the DNA stability of a mammalian host cell. One such vector was generated by cloning a mutator gene, which for purposes of this application, employed the dominant negative mismatch repair protein (PMS134) into a fusion transcription vector containing the negative selection marker HSV-TK. The vector of the invention appears to encode a novel function, since the expression of the encoded mutator protein can generate genetically diverse pools of cells that can be selected for novel phenotypes. Once a subline exhibiting a novel phenotype is derived, it can be expanded and selected for subclones that no longer express the mutator gene via a selection using a fused negative selection marker.

Characterization of Selectable Mutator Fusion Genes:

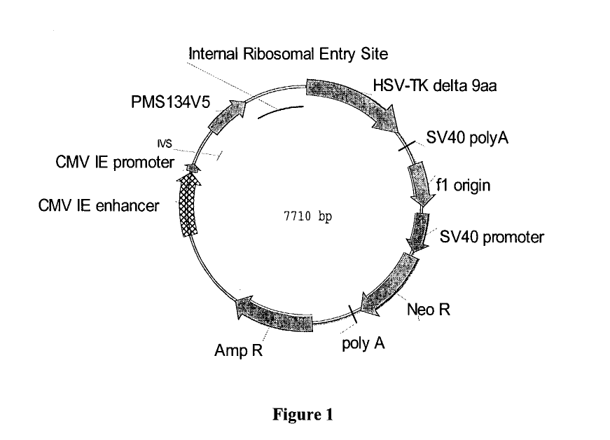

Mutator expression vectors containing a PMS134 (SEQ ID NO: 2) cDNA fused to an IRES sequence followed by a negative selection marker gene consisting of a modified HSV-TK gene (SEQ ID NO: 1) were generated that are capable of producing proteins from each of these genes (referred to as PMS 134-TK). The pIRES-PMS134-TK expression vector (FIG. 1) containing this gene was introduced into HEK293 cells. Robust expression of each gene could be detected in HEK293 cells when the fusion transcript was cloned into an expression cassette under control of the cytomegalovirus (CMV) promoter, followed by the SV40 polyadenylation signal. In addition, the vector contained a neomycin selectable marker (NEO) that allowed for positive selection of transfected cells containing the PMS134-TK gene. The vector of the invention facilitates the regulated expression of a recombinant mutator protein driven by a promoter/enhancer region to which it is linked through the use of negative selection.

Moreover, additional negative selection markers can be used to regulate mutator genes expression in mammalian cells as described herein, as can other mutator gene from other types of cells or from variants of mammalian derived homologs be used such as those described by Woods et al. (2001). In addition, it is known in the art that other genes involved in DNA repair can cause a mutator phenotype. Thus, the DNA fragments described and claimed herein also include fragments that encode for such genes can be used.

Other types of fusion fragments described herein can also be developed, for example, fusion genes comprising a mutator gene and a negative selection marker, formed, for example, by juxtaposing the mutator gene open reading frame (ORF) in-frame with the negative selection marker, or positioning the negative selection marker first, followed by the mutator gene. Such combinations can be contiguously linked or arranged to provide optimal spacing for expressing the two gene products (i.e., by the introduction of “spacer” nucleotides between the gene ORFs). Regulatory elements can also be arranged to provide optimal spacing for expression of both genes such as the use of an IRES motif.

The vector disclosed herein was engineered using the cDNA comprised of the human PMS134 (SEQ ID NO: 2) mutator allele and a modified HSV-TK gene (SEQ ID NO: 1). Modified changes to the HSV-TK were made by polymerase chain reaction using the nucleotide sequence set forth in sense primer (5′-ttttctagaccatggcgtctgcgttcg-3′ SEQ ID NO: 7) which consists of a restriction site to facilitate cloning and a Kozak consensus sequence for efficient translation; and the antisense primer (5′-tttgcggccgctacgtgtttcagttagcctcc-3′ SEQ ID NO: 8) by site-directed mutagenesis techniques that are known in the art.

The expression of recombinant proteins is driven by an appropriate eukaryotic promoter/enhancer and the inventive fusion transcript. Cells are transfected with a plasmid selected under low stringency for the dominant selectable marker and then screened for mutator gene expression. Mutator positive cells are expanded to enhance mutagenesis and selection for a desired phenotype.

Inclusion of an IRES sequence into fusion vectors may be beneficial for enhancing expression of some proteins. The IRES sequence appears to stabilize expression of the genes under selective pressure (Kaufman et al. 1991). For some polypeptides, the IRES sequence is not necessary to achieve high expression levels of the downstream sequence.

Expression Vectors:

Recombinant expression vectors include synthetic or cDNA-derived DNA fragments encoding a mutator protein and a dominant negative selection marker, operably linked to suitable regulatory elements derived from mammalian, viral, or insect genes. Such regulatory elements include a transcriptional promoter, sequences encoding suitable mRNA ribosomal binding sites, and sequences, which control the termination of transcription and translation. Mammalian expression vectors may also comprise nontranscribed elements such as an origin of replication, a suitable promoter and enhancer linked to the gene to be expressed, other 5′ or 3′ flanking nontranscribed sequences, 5′ or 3′ nontranslated sequences such as necessary ribosome binding sites, a polyadenylation site, splice donor and acceptor sites, and transcriptional termination sequences. An origin of replication that confers the ability to replicate in a host, and a selectable gene to facilitate recognition of transformants, may also be incorporated. In addition, the expression vector consists of a positive selectable marker that allows for selection of recipient hosts that have taken up the expression vector.

The transcriptional and translational control sequences in expression vectors to be used in transforming vertebrate cells may be provided by viral sources. For example, commonly used promoters and enhancers are derived from human CMV, Adenovirus 2, Simian Virus 40 (SV40), and Polyoma. Viral genomic promoters, control and/or signal sequences may be utilized to drive expression which are dependent upon compatible host cells. Exemplary vectors can be constructed such as described by Okayama and Berg ((1983) Mol. Cell. Biol. 3:280). Promoters derived from house keeping genes can also be used (i.e., the β-globin and the EF-1α promoters), depending on the cell type in which the vector is to be expressed as described (Klehr et al. (1991); Grosveld, et al., (1987)).

Expression vectors containing internal ribosome entry sites (IRES) used for the expression of multiple transcripts have been described previously (Kim S. K. and B. J. Wold (1985) Cell 42:129; Kaufman et al. (1991); Mosley et al. (1989) Cell; Subramani et al., (1981) Mol. Cell. Biol. 1:854). Other RES-based vectors include those such as the pCDE vector, which contains the IRES derived from the murine encephalomyocarditis virus (Jang and Wimmer, (1990) Genes and Dev. 4:1560), which is cloned between the adenovirus tripartite leader and a DHFR cDNA. Other types of expression vectors will also be useful for regulated expression of mutator genes through negative selection such as those described in U.S. Pat. Nos. 4,634,665 to Axel et al. and 4,656,134 to Ringold et al.

Sequences of the modified HSV-TK gene and examples of mutator genes that can be used for enhancing genetic hypermutability include the following:

Morphotek HSV-TK cDNA (double stranded sequence)

(SEQ ID NO:1)

ATG GCT TCG TAC CCC TGC CAT CAA CAC GCG TCT GCG TTC GAC CAG GCT GCG CGT TCT CGC
TAC CGA AGC ATG GGG ACO GTA GTT GTG CGC AGA CGC AAG CTG GTC CGA CGC GCA AGA GCG
GGC CAT AGC AAC CGA CGT ACG GCG TTG CCC CCT CGC CGG CAG CAA GAA CCC ACG CPA CTC
CCG OTA TCG TTG CCT CCA TGC CCC AAC CCG CCA GCG GCC GTC CTT CTT CGG TGC CTT CAC
CCC CTG GAG CAG AAA ATG CCC ACG CTA CTG CCC GTT TAT ATA GAC GGT CCT CAC CCC ATG
GCG GAC CTC GTC TTT TAC GGG TGC GAT GAC GCC CPA ATA TAT CTG CCA GGA GTG CCC TAC
GGG AAA ACC ACC ACC ACG CPA CTG CTG GTG CCC CTG GGT TCG CCC GAC GAT ATC GTC TAC
CCC TTT TGG TCG TGG TGC GTT GAC GAC CAC CCG GAC CCA AGC GCG CTG CTA TAG CAG ATG
GTA CCC GAG CCG ATG ACT TAC TGG CAG GTG CTG GGG GCT TCC GAG ACA ATC GCG AAC ATC
CAT GGG CTC GOC TAC TGA ATG ACC GTC CAC GAC CCC CGA AGG CTC TGT TAG CGC TTG TAG
TAC ACC ACA CAA CAC CGC CTC GAC CAG GGT GAG ATA TCG GCC GGG GAC GCG GCG GTG GTA
ATG TGG TGT GTT GTG GCG GAG CTG GTC CCA CTC TAT AGC CGG CCC CTG CGC CGC CAC CAT
ATG ACA AGC GCC CAG ATA ACA ATG GGC ATG CCT TAT GCC GTG ACC GAC GCC GTT CTG GCT
TAC TGT TCG CGG GTC TAT TGT TAC CCG TAC GGA ATA CGG CAC TGG CTG CGG CAA GAC CGA
CCT CAT ATC GGG GGG GAG GCT GGG AGC TCA CAT GCC CCG CCC CCG GCC CTC ACC CTC ATC
GGA GTA TAG CCC CCC CTC CGA CCC TCG AGT GTA CGG GGC GGG GGC CGG GAG TGG GAG TAG
TTC GAC CGC CAT CCC ATC GCC GCC CTC CTG TGC TAC CCG GCC GCG CGG TAC CTT ATG GGC
AAG CTG GCG GTA GGG TAG CGG CGG GAG GAC ACG ATG GGC CGG CGC GCC ATG GAA TAC CCG
AGC ATG ACC CCC CAG GCC GTG CTG GCG TTC GTG GCC CTC ATC CCG CCG ACC TTG CCC GGC
TCG TAC TGG GGG GTC CGG CAC GAC CGC AAG CAC CGG GAG TAG GGC GGC TGG AAC GGG CCG
ACC AAC ATC GTG CTT GGG GCC CTT CCG GAG GAG AGA CAC ATC GAC CGC CTG GCC AAA CGC
TGG TTG TAG CAC GAA CCC CGG GAA GGC CTC CTG TCT GTG TAG CTG GCG GAC CGG TTT GCG
CAG CGC CCC GGC GAG CGG CTG GAC CTG GCT ATG CTG GCT GCG ATT CGC CGC GTT TAC GGG
GTC GCG GGG CCG CTC GCC GAC CTG GAC CGA TAC GAC CGA CGC TAA GCG GCG CAA ATG CCC
CTA CTT GCC AAT ACG GTG CGG TAT CTG CAG GGC GGC GGG TCG TGG CGG GAG GAT TGG GGA
GAT GAA CGG TTA TGC CAC GCC ATA GAC GTC CCG CCG CCC AGC ACC GCC CTC CTA ACC CCT
GAG CTT TCG GGG ACG GCC GTG CCG CCC CAG GGT GCC GAG CCC CAG AGC AAC GCG GGC CCA
GTC GAA AGC CCC TGC CGG CAC GGC GGG GTC CCA CGG CTC GGG GTC TCG TTG CGC CCG GGT
CGA CCC CAT ATC GGG GAC ACG TTA TTT ACC CTG TTT CGG GCC CCC GAG TTG CTG GCC CCC
GCT GGG GTA TAG CCC CTG TGC AAT AAA TGG GAC AAA GCC CGG GGG CTC AAC GAC CGG GGG
AAC GGC GAC CTG TAT AAC GTG TTT GCC TGG GCC TTG GAC GTC TTG GCC AAA CGC CTC CGT
TTG CCG CTG GAG ATA TTG CAC AAA CGG ACC CGG AAC CTG CAG AAC CGG TTT GCG GAG GCA
CCC ATG CAC GTC TTT ATC CTG GAT TAC GAC CAA TCG CCC GCC GGC TAC CGG GAC GCC CTG
GGG TAC GTG CAG AAA TAG GAC CTA ATG CTG GTT AGC GGG CGG CCG ATG GCC CTG CGG GAC
CTG CAA CTT ACC TCC GGG ATG GTC CAG ACC CAC GTC ACC ACC CCA GGC TCC ATA CCG ACG
GAC GTT GAA TGG AGG CCC TAC CAG GTC TGG GTG CAG TGG TGG GGT CCG AGG TAT GGC TGC
ATC TGC GAC CTG GCG CGC ACG TTT GCC CGG GAG ATG GGG GAG GCT AAC TGA AAC ACG GAA
TAG ACG CTG GAC CGC GCG TGC AAA CGG GCC CTC TAC CCC CTC CGA TTG ACT TTG TGC CTT
GGA GAC AAT ACC GGA AGG AAC CCG CGC TAT GAC GGC AAT AAA AAG ACA GAA TAA AAC GCA
CCT CTG TTA TGG CCT TCC TTG GGC GCG ATA CTG CCG TTA TTT TTC TGT CTT ATT TTG CGT
CGG GTG TTG GOT CGT TTG TTC ATA AAC GCG GGG TTC GGT CCC AGG GCT GGC A
GCC CAC AAC CCA GCA AAC AAG TAT TTG CGC CCC AAG CCA GGG TCC CGA CCG T

Morphotek HSV-TK protein

(SEQ ID NO:11)

| | |
|---|---|
| 1 | MTYWXVLGAS ETIANIYTTQ HRLDQGEISA GDAAVVXTSA QITMGMPYAV TDAVLAPHIG |
| 61 | GEAGSSHAPP PALTLIFDRH PIAALLCYPA ARYLMGSMTP QAVLAFVALI PPTLPGTNIV |
| 121 | LGALPEDRHI DRLAKRQRPG ERLDLAMLAA IRRVYGLLAN TVRYLQCGGS WREDWGQLSG |
| 181 | TAVPPQGAEP QSNAGPRPHI GDTLFTLFRA PELLAPNGDL YNVFAWALDV LAKRLRSMHV |
| 241 | FILDYDQSPA GCRDALLQLT SGMVQTHVTT PGSIPTICDL ARTFAREMGE AN |

hPMS2-134 (human cDNA) (SEQ ID NO: 2), hPMS2-134 (human protein) (SEQ ID NO: 12); PMS2 (human protein) (SEQ ID NO: 3); PMS2 (human cDNA) (SEQ ID NO: 13); PMS1 (human protein) (SEQ ID NO: 4); PMS1 (human cDNA) (SEQ ID NO: 14); MSH2 (human protein) (SEQ ID NO: 5); MSH2 (human cDNA) (SEQ ID NO: 15); MLH1 (human protein) (SEQ ID NO: 6); MLH1 (human cDNA) (SEQ ID NO: 16); PMSR2 (human cDNA Accession No. U38964) (SEQ ID NO: 17); hPMSR2 (human protein, Accession No. U38964) (SEQ ID NO: 18); HPMSR3 (human cDNA, Accession No. U38979) (SEQ ID NO: 19); hPMSR3 (human protein, Accession No. U38979) (SEQ ID NO: 20); MLH3 (human protein, Accession No. AAF23904) (SEQ ID NO: 21); MLH3 (human cDNA, Accession No. AF195657) (SEQ ID NO: 22).

Host Cells

Transformed host cells are cells which have been transfected with expression vectors generated by recombinant DNA techniques and which contain sequences encoding the fusion transcript containing the mutator gene and negative selection marker. Various mammalian cell culture systems can be employed to express recombinant protein. Examples of suitable mammalian host cell lines include the COS-7 lines of monkey kidney cells, as described by Gluzman (1981) Cell 23:175), and other cell lines capable of expressing an appropriate vector including but not limited to HEK293 (Nicolaides et al. 1998), T98G, CV-1/EBNA, L cells (Holst et al. (1988)), C127, 3T3, Chinese hamster ovary (CHO) (Weidle, et al. (1988)), HeLa TK-ts13 (Nicolaides et al. (1998)), NS1, Sp2/0 myeloma cells and BHK cell lines.

Transfection and Generation of Stable Lines:

In general, transfection will be carried out using a suspension of cells, or a single cell, but other methods can also be applied as long as a sufficient fraction of the treated cells or tissue incorporates the polynucleotide so as to allow transfected cells to be grown and utilized. Techniques for transfection are well known. Several transformation protocols are known in the art, [see Kaufman, R. J. (1988) Meth. Enzymology 185:537]. The transformation protocol employed will depend on the host cell type and the nature of the gene of interest. The basic requirements of any such protocol are first to introduce DNA encoding the protein of interest into a suitable host cell, and then to identify and isolate host cells which have incorporated the vector DNA in a stable, expressible manner. Available techniques for introducing polynucleotides include but are not limited to electroporation, transduction, cell fusion, the use of calcium chloride, and packaging of the polynucleotide together with lipid for fusion with the cells of interest. If the transfection is stable, such that the selectable marker gene is expressed at a consistent level for many cell generations, then a cell line results.

One common method for transfection into mammalian cells is calcium phosphate precipitation as described by Nicolaides et al. (1998). Another method is polyethylene glycol (PEG)-induced fusion of bacterial protoplasts with mammalian cells (Schaffner et al. (1980) Proc. Natl. Acad. Sci. USA 77:2163). Yet another method is electroporation. Electroporation can also be used to introduce DNA directly into the cytoplasm of a host cell, for example, as described by Potter et al. (1988) Proc. Natl. Acad. Sci. USA 81:7161.

Transfection of DNA can also be carries out using polyliposome reagents such as Lipofectin and Lipofectamine (Gibco BRL, Gaithersburg, Md.) which form lipid-nucleic acid complexes (or liposomes) which, when applied to cultured cells, facilitate uptake of the nucleic acid into the cells.

Once a cell is transfected, pools are selected to identify cells that have taken up the expression vector. Useful dominant selectable markers include microbially derived antibiotic resistance genes, for example neomycin, kanamycin, tetracycline, hygromycin, and penicillin resistance.

The transfected cells may be selected in a number of ways. For cells in which the vector also contains an antibiotic resistance gene, the cells may be selected for antibiotic resistance, which positively selects for cells containing the vector. In other embodiments, the cells may be allowed to mutate, or may be further treated with a mutagen to enhance the rate of mutation and selected based on the presence of an altered phenotype in a gene of interest. Once a phenotype of interest is achieved, the cells may be negatively selected based on the HSV-TK gene such that a genetically stable cell is obtained containing the altered phenotype in the gene of interest.

For further information on the background of the invention the following references may be consulted, each of which is incorporated herein by reference in its entirety:

References

1) Nicolaides, N. C. et al. (1998) A naturally occurring hPMS2 mutation can confer a dominant negative mutator phenotype. Mol. Cell. Biol. 18:1635-1641.

2) Perucho, M. (1996) Cancer of the microsatellite mutator phenotype. Biol. Chem. 377:675-684.

3) Nicolaides, N. C. et al. (1995) Genomic organization of the human PMS2 gene family. Genomics 30:195-206.

4) Nicolaides, N. C. et al. (1997) Interleukin 9: a candidate gene for asthma. Proc. Natl. Acad. Sci. USA 94:13175-13180.

5) Grasso, L. et al. (1998) Molecular analysis of human interleukin-9 receptor transcripts in peripheral blood mononuclear cells. Identification of a splice variant encoding for a nonfunctional cell surface receptor. J. Biol. Chem. 273:24016-24024.

6) Grosveld, F. et al. (1987) Position-independent, high-level expression of the human β-globin gene in transgenic mice. Cell 51:975-985.

7) Holst, A. et al. (1988) Murine genomic DNA sequences replicating autonomously in mouse L cells. Cell 52:355-365.

8) Kaufman, R. et al. (1991) Improved vectors for stable expression of foreign genes in mammalian cells by use of the untranslated leader sequence from EMC virus. Nucl. Acids Res. 19(16):4485-4490, 1991.

9) Klehr, D. et al. (1991) Scaffold-attached regions from the human interferon β domain can be used to enhance the stable expression of genes under the control of various promoters. Biochemistry 30:1264-1270.

10) Lipkin, S. M., et al. (2000) MLH3: a DNA mismatch repair gene associated with mammalian microsatellite instability. Nat. Genet. 24(1):27-35.

11) McBratney, S. et al. (1993) Internal initiation of translation. Curr. Opin. Cell Biol. 5:961-965.

12) Wegner, M. et al. (1990) Interaction of a protein with a palindromic sequence from murine rDNA increases the occurrence of amplification-dependent transformation in mouse cells. J. Biol. Chem. 265(23):13925-13932.

13) Weidle, U. et al. (1988) Amplified expression constructs for human tissue-type plasminogen activator in Chinese hamster ovary cells: instability in the absence of selective pressure Gene 66:193-203.

The above disclosure generally describes the present invention. A more complete understanding can be obtained by reference to the following specific examples, which are provided herein for purposes of illustration only, and are not intended to limit the scope of the invention.

### EXAMPLES

### Example 1

### Engineering the Mutator/Negative Selection Fusion

To demonstrate the utility of mutator/negative selection marker fusions to regulate genomic stability of a host cell, the pIRES-PMS134-TK vector was constructed. The pCMV vector containing the CMV promoter followed by a multiple polylinker cloning site and an SV40 polyadenylation signal was used as a backbone along with a constitutively expressed neomycin phosphotransferase gene as a dominant selectable marker. The pCMV cassette contained an internal ribosome entry site (IRES) from the encephalomyocarditis virus (ECMV) that was cloned within the polylinker region. The gene encoding the PMS134 dominant negative mismatch repair cDNA, when expressed in an otherwise mismatch repair (MMR) proficient cell, renders these host cells MMR deficient [Nicolaides, N. C. et al. (1998) Mol. Cell. Biol. 18:1635-1641, U.S. Pat. No. 6,146,894 to Nicolaides et al.] The PMS134 gene was cloned into the EcoRI-XbaI site located upstream of the IRES sequence. The PMS134 cDNA was modified to facilitate the cloning of the insert as well as to contain a small sequence tag at the C-terminus of the PMS134 polypeptide. The fragment was engineered by PCR using the sense primer (5′-TAATGAATTCACGACTCACTATAGGG-3′, SEQ ID:NO 9) that contained an EcoRI site and the antisense primer (5′-TTTGAATTCTCACTACGTAGAATCGAGACCGAG-3′ SEQ ID NO: 10) that included sequence encoding for the V5 polypeptide fused in-frame with the PMS134 cDNA after residue 133 followed by two termination codons and an EcoRI restriction site. The PMS134 plasmid (Nicolaides et al., 1998) was used as template. PCR products containing the modified PMS134 was digested with EcoRI and cloned into the EcoRI site upstream of the IRES sequence. A modified HSV-TK gene was inserted downstream of the IRES sequence. HSV-TK was modified using a sense (SEQ ID: NO 7) and antisense primer (SEQ ID:NO 8) primer that resulted in the creation of XbaI-NotI fragments that were generated by PCR, digested and subcloned into the expression cassette. A diagram showing the vector, referred to as pIRES-PMS134-TK is given in FIG. 1.

FIG. 1: Schematic pf pIRES-PMS134-TK. (AmpR)=Ampicillin resistance gene; (NeoR)=neomycin phosphotransferase gene fused at the C-terminus; (PMS134V5)=PMS134 mismatch repair dominant negative gene with a V5 epitope; (HSV-TK delta9aa)=modified herpes simplex virus thymidine kinase gene; (IVS)=intervening sequence from the rabbit β-globin gene; (f1 origin)=bacterial origin of replication (see FIG. 1).

### EXAMPLE 2

### Generation of Negatively Selected Mutator Subclones

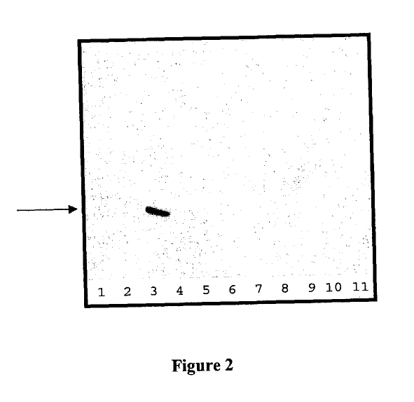

To demonstrate the ability to use a mutator expression vector fused to a negative selection marker as a means to regulate genomic stability of a host cell, the pIRES-PMS134-TK plasmid was transfected into a MMR proficient human cell line (HEK293). Cells were also transfected with empty vector as a negative control. Cells were transfected using lipofectamine following the manufacturer's recommendations. Transfected pools were selected for 10-14 days in 0.4 mg/ml of G418 (neomycin analog) to select for clones containing the expression vector. Next, cells are analyzed for gene expression via western blot. Briefly, 50,000 cells from the PMS134-TK culture or controls are centrifuged, and resuspended in 150 μls of 2× SDS buffer and cultures were analyzed for PMS134 protein expression by western blot. Western blots were carried out as follows. 50 μls of each PMS134 or empty vector culture was directly lysed in 2× lysis buffer (60 mM Tris, pH 6.8, 2% SDS, 10% glycerol, 0.1 M 2-mercaptoethanol, 0.001% bromophenol blue) and samples were boiled for 5 minutes. Lysate proteins were separated by electrophoresis on 4-20% Tris glycine gels (Novex). Gels were electroblotted onto Immobilon-P (Millipore) in 48 mM Tris base, 40 mM glycine, 0.0375% SDS, 20% methanol and blocked overnight at 4° C. in Tris-buffered saline plus 0.05% Tween-20 and 5% dry milk. Filters were probed with a monoclonal antibody generated against the V5 fusion polypeptide located at the C-terminus of the PMS134 protein, followed by a secondary goat anti-mouse horseradish peroxidase-conjugated antibody. After incubation with the secondary antibody, blots are developed using chemiluminescence (Pierce) and exposed to film to measure PMS134 expression. As shown in FIG. 2, a robust expression of PMS134 could be detected in cells containing pIRES-PMS134-TK (FIG. 2, lane 3) in contrast to cells expressing empty vector (FIG. 2, lane 2), which had no signal.

Next, selected clones expressing PMS134 are expanded and analyzed for MMR deficiency using methods as previously described. Briefly, cells containing the pIRES-PMS134-TK vector or control were measured for microsatellite instability of endogenous loci using the BAT26 diagnostic marker as described (Hoang J. M. et al. (1997) Cancer Res. 57:300-303). Cells are harvested and genomic DNA is isolated using the salting out method (Nicolaides, N. C. et al. (1991) Mol. Cell. Biol. 11:6166-6176). DNAs are diluted by limiting dilution to determine the level of sensitivity of the assay. DNAs are PCR amplified using the BAT26F: 5′-tgactacttttgacttcagcc-3′ (SEQ ID NO: 25) and the BAT26R: 5′-aaccattcaacatttttaaccc-3′ (SEQ ID NO: 26) primers in buffers as described (Nicolaides, N. C. et al. (1995) Genomics 30:195-206). Briefly 1 pg to 100 ngs of DNA is amplified using the following conditions: 94° C. for 30 sec, 58° C. for 30 sec, 72° C. for 30 sec for 30 cycles. PCR reactions are electrophoresed on 12% polyacrylamide TBE gels (Novex) or 4% agarose gels and stained with ethidium bromide.

To measure for microsatellite stability in 293 cells, DNAs were amplified using the reaction conditions above. No altered alleles were found in the MMR-proficient control cells as expected since MI only occurs in MMR defective cell hosts, while a number of alterations can be found in PMS134 expressing cells (Perucho, M. (1996)). The results showed that the PMS134 mutant could exert a robust dominant negative effect, resulting in biochemical and genetic manifestations of MMR deficiency.

Once a hypermutable cell line is developed, it can be screened for to identify subclones with novel phenotypes for target discovery and/or product development. Once the subclone with a desired phenotype is identified, it is important to be able to restore the DNA stability within the host cell. In order to accomplish this, one must remove or completely suppress the expression of the mutator gene. Briefly, cultures are grown in the presence of the prodrug ganciclovir (Sigma), which kills cells expressing the HSV-TK gene product for 5 days. After 5 days, cells are grown for 10 days in growth media alone at which time greater than 95% of cells die off. Resistant clones are then picked and expanded in 10 cm petri dishes. Cells are grown for 3 weeks and then a portion is analyzed for PMS134 expression by western blot and RT-PCR. FIG. 2 demonstrates a typical result observed in the ganciclovir-resistant cells (GR) whereas robust expression is observed in the untreated PMS134-TK cultures (FIG. 2, lane 3), none was observed in the GR resistant cells (FIG. 2, lanes 4-11). Analysis of cells after two months of culture have not observed re-expression of the PMS 134 gene using western or RT-PCR analysis (not shown). Finally, to demonstrate that genomic stability is restored, cells are analyzed for microsatellite instability as described above. No de novo instable microsatellites are observed in the GR cells in contrast to the parental IRES-PMS134-TK line as expected.

FIG. 2 shows expression of PMS134 in negatively selected, ganciclovir resistant HEK293 clones. Wild type (lane 1), empty vector (lane 2), IRES-PMS134-TK pool (lane 3) or IRES-PMS134-TK ganciclovir-resistant selected subclones (lanes 4-11). HEK293 cells were analyzed by western blot for PMS134 expression using an anti-V5 probe that can recognize the PMS134-V5 fusion protein generated by the IRES-PMS134-TK vector as shown in lane 3. Lanes 4-11 are lysates from IRES-PMS134-TK cells negatively selected for expression of the PMS134-HSV-TK fusion transcript after 5 days of ganciclovir exposure. The arrow indicates polypeptide of expected molecular weight.

Discussion

The results and observation described here lead to several conclusions. First, expression of the PMS134-HSV-TK can generate a mutator phenotype in mammalian cells. Second, pooled cultures of cells containing this expression construct can be negatively selected to restore the genetic stability of the host. These findings teach the use of a vector system suitable for generating stable altered lines for discovery and development using mutator alleles linked to a negative selection marker that can completely inhibit the expression of a mutator expression vector.

What is claimed is:

1. A polynucleotide vector comprising a constitutively active promoter operatively linked to a sequence encoding a dominant negative allele of a mismatch repair gene, an internal ribosome entry site -and a negative selection marker sequence.

2. The vector of claim 1 wherein said mismatch repair gene encodes a polypeptide selected from the group consisting of SEQ ID NO: 3, SEQ ID NO: 4, SEQ ID NO: 5, SEQ ID NO: 6, SEQ ID NO: 12, SEQ ID NO: 18, SEQ ID NO: 20, and SEQ ID NO: 21.

3. The vector of claim 1 wherein said mismatch repair gene comprises a nucleotide sequence selected from the group consisting of SEQ ID NO: 2, SEQ ID NO: 13, SEQ ID NO: 14, SEQ ID NO: 15, SEQ ID NO: 16, SEQ ID NO: 17, SEQ ID NO: 19 and SEQ ID NO: 20.

4. The vector of claim 1 wherein said dominant negative allele of a mismatch repair gene is PMS134.

5. The vector of claim 1 further comprising a polyadenylation signal downstream of said selection marker sequence.

6. The vector of claim 1 wherein said negative selection marker is an HSV-TK gene.

7. The vector of claim 6 wherein said HSV-TK gene comprises the nucleotide sequence of SEQ ID NO: 1.

8. The vector of claim 1 wherein said promoter is selected from the group consisting of a viral promoter including CMV promoter, an adenovirus 2 promoter, an SV40 promoter, and a polyoma promoter.

9. The vector of claim 1 wherein said promoter is selected for host specific expression using constitutively active housekeeping promoters from a host cells genome.

10. The vector of claim 1 further comprising a sequence encoding a selectable marker for transfection.

11. The vector of claim 10 wherein said selectable marker for transfection is an antibiotic resistance gene.

12. The vector of claim 11 wherein said antibiotic resistance gene is selected from the group consisting of a neomycin resistance gene, a hygromycin resistance gene, a kanamycin resistance gene, a tetracycline resistance gene, and a penicillin resistance gene.

13. The vector of claim 1 wherein said dominant negative allele of a mismatch repair gene encodes a PMS2 homolog comprising the amino acid sequence of SEQ ID NO: 23 or SEQ NO: 24.

14. A transformed host cell comprising a vector according to claim 1.

15. The host cell according to claim 14, wherein said host cell is a eukaryotic cell.

16. A method for producing an isolated, genetically stable cell with a new phenotype, the method comprising the steps of:
a) culturing a recombinant host cell according to claim 14 under conditions for the expression of the polypeptide rendering the cell hypermutable thereby producing a library of cells;
b) selecting for clones from the cell library exhibiting new phenotypes whereby positive clones are expanded and propagated; and
c) negatively selecting for clones no longer expressing the mutator gene rendering the resulting subclones genetically stable.

17. The method of claim 16 wherein said cell is a mammalian cell.

18. The method of claim 16 wherein said cell is a plant cell.

17. 19. The method of claim 16 wherein said cell is an amphibian cell.

20. The method of claim 16 wherein said cell is an insect cell.

21. The method of claim 16 wherein said cell is a fungal cell.

22. The method of claim 16 further comprising treating said cells with a mutagen during said culturing step.


SEQUENCE LISTING (26 sequences)

SEQ ID NO: 1 - DNA, 1252 bp, Artificial Sequence
  Vector Sequence
atggcttcgtacccctgccatcaacacgcgtctgcgttcgaccaggctgcgcgttctcgcggccatagcaaccgacgtacggcgttgcgccctcgccggcagcaagaagccacggaagtccgcctggagcagaaaatgcccacgctactgcgggtttatatagacggtcctcacgggatggggaaaaccaccaccacgcaactgctggtggccctgggttcgcgcgacgatatcgtctacgtacccgagccgatgacttactggcaggtgctgggggcttccgagacaatcgcgaacatctacaccacacaacaccgcctcgaccagggtgagatatcggccggggacgcggcggtggtaatgacaagcgcccagataacaatgggcatgccttatgccgtgaccgacgccgttctggctcctcatatcgggggggaggctgggagctcacatgccccgcccccggccctcaccctcatcttcgaccgccatcccatcgccgccctcctgtgctacccggccgcgcggtaccttatgggcagcatgaccccccaggccgtgctggcgttcgtggccctcatcccgccgaccttgcccggcaccaacatcgtgcttggggcccttccggaggacagacacatcgaccgcctggccaaacgccagcgccccggcgagcggctggacctggctatgctggctgcgattcgccgcgtttacgggctacttgccaatacggtgcggtatctgcagggcggcgggtcgtggcgggaggattggggacagctttcggggacggccgtgccgccccagggtgccgagccccagagcaacgcgggcccacgaccccatatcggggacacgttatttaccctgtttcgggcccccgagttgctggcccccaacggcgacctgtataacgtgtttgcctgggccttggacgtcttggccaaacgcctccgtcccatgcacgtctttatcctggattacgaccaatcgcccgccggctaccgggacgccctgctgcaacttacctccgggatggtccagacccacgtcaccaccccaggctccataccgacgatctgcgacctggcgcgcacgtttgcccgggagatgggggaggctaactgaaacacggaaggagacaataccggaaggaacccgcgctatgacggcaataaaaagacagaataaaacgcacgggtgttgggtcgtttgttcataaacgcggggttcggtcccagggctggca

SEQ ID NO: 2 - DNA, 426 bp, Homo sapiens
cgaggcggatcgggtgttgcatccatggagcgagctgagagctcgagtacagaacctgctaaggccatcaaacctattgatcggaagtcagtccatcagatttgctctgggcaggtggtactgagtctaagcactgcggtaaaggagttagtagaaaacagtctggatgctggtgccactaatattgatctaaagcttaaggactatggagtggatcttattgaagtttcagacaatggatgtggggtagaagaagaaaacttcgaaggcttaactctgaaacatcacacatctaagattcaagagtttgccgacctaactcaggttgaaacttttggctttcggggggaagctctgagctcactttgtgcactgagcgatgtcaccatttctacctgccacgcatcggcgaaggttggaacttga

SEQ ID NO: 3 - protein, 932 aa, Homo sapiens
MKQLPAATVRLLSSSQIITSVVSVVKELIENSLDAGATSVDVKLENYGFDKIEVRDNGEGIKAVDAPVMAMKYYTSKINSHEDLENLTTYGFRGEALGSICCIAEVLITTRTAADNFSTQYVLDGSGHILSQKPSHLGQGTTVTALRLFKNLPVRKQFYSTAKKCKDEIKKIQDLLMSFGILKPDLRIVFVHNKAVIWQKSRVSDHKMALMSVLGTAVMNNMESFQYHSEESQIYLSGFLPKCDADHSFTSLSTPERSFIFINSRPVHQKDILKLIRHHYNLKCLKESTRLYPVFFLKIDVPTADVDVNLTPDKSQVLLQNKESVLIALENLMTTCYGPLPSTNSYENNKTDVSAADIVLSKTAETDVLFNKVESSGKNYSNVDTSVIPFQNDMHNDESGKNTDDCLNHQISIGDFGYGHCSSEISNIDKNTKNAFQDISMSNVSWENSQTEYSKTCFISSVKHTQSENGNKDHIDESGENEEEAGLENSSEISADEWSRGNILKNSVGENIEPVKILVPEKSLPCKVSNNNYPIPEQMNLNEDSCNKKSNVIDNKSGKVTAYDLLSNRVIKKPMSASALFVQDHRPQFLIENPKTSLEDATLQIEELWKTLSEEEKLKYEEKATKDLERYNSQMKRAIEQESQMSLKDGRKKIKPTSAWNLAQKHKLKTSLSNQPKLDELLQSQIEKRRSQNIKMVQIPFSMKNLKINFKKQNKVDLEEKDEPCLIHNLRFPDAWLMTSKTEVMLLNPYRVEEALLFKRLLENHKLPAEPLEKPIMLTESLFNGSHYLDVLYKMTADDQRYSGSTYLSDPRLTANGFKIKLIPGVSITENYLEIEGMANCLPFYGVADLKEILNAILNRNAKEVYECRPRKVISYLEGEAVRLSRQLPMYLSKEDIQDIIYRMKHQFGNEIKECVHGRPFFHHLTYLPETT

SEQ ID NO: 4 - protein, 932 aa, Homo sapiens; identical to SEQ ID NO: 3

SEQ ID NO: 5 - protein, 934 aa, Homo sapiens [sequence omitted]

SEQ ID NO: 6 - protein, 756 aa, Homo sapiens
MSFVAGVIRRLDETVVNRIAAGEVIQRPANAIKEMIENCLDAKSTSIQVIVKEGGLKLIQIQDNGTGIRKEDLDIVCERFTTSKLQSFEDLASISTYGFRGEALASISHVAHVTITTKTADGKCAYRASYSDGKLKAPPKPCAGNQGTQITVEDLFYNIATRRKALKNPSEEYGKILEVVGRYSVHNAGISFSVKKQGETVADVRTLPNASTVDNIRSIFGNAVSRELIEIGCEDKTLAFKMNGYISNANYSVKKCIFLLFINHRLVESTSLRKAIETVYAAYLPKNTHPFLYLSLEISPQNVDVNVHPTKHEVHFLHEESILERVQQHIESKLLGSNSSRMYFTQTLLPGLAGPSGEMVKSTTSLTSSSTSGSSDKVYAHQMVRTDSREQKLDAFLQPLSKPLSSQPQAIVTEDKTDISSGRARQQDEEMLELPAPAEVAAKNQSLEGDTTKGTSEMSEKRGPTSSNPRKRHREDSDVEMVEDDSRKEMTAACTPRRRIINLTSVLSLQEEINEQGHEVLREMLHNHSFVGCVNPQWALAQHQTKLYLLNTTKLSEELFYQILIYDFANFGVLRLSEPAPLFDLAMLALDSPESGWTEEDGPKEGLAEYIVEFLKKKAEMLADYFSLEIDEEGNLIGLPLLIDNYVPPLEGLPIFILRLATEVNWDEEKECFESLSKECAMFYSIRKQYISEESTLSGQQSEVPGSIPNSWKWTVEHIVYKALRSHILPPKHFTEDGNILQLANLPDLYKVFERC

SEQ ID NO: 7 - DNA, 27 bp, Artificial Sequence
  Oligonucleotide Primer
ttttctagaccatggcgtctgcgttcg

SEQ ID NO: 8 - DNA, 32 bp, Artificial Sequence
  Oligonucleotide Primer
tttgcggccgctacgtgtttcagttagcctcc

SEQ ID NO: 9 - DNA, 26 bp, Artificial Sequence
  Oligonucleotide Primer
taatgaattcacgactcactataggg

SEQ ID NO: 10 - DNA, 33 bp, Artificial Sequence
  Oligonucleotide Primer
tttgaattctcactacgtagaatcgagaccgag

SEQ ID NO: 11 - protein, 292 aa, Artificial Sequence
  human PMS2herpes simplex TK chimera
MTYWXVLGASETIANIYTTQHRLDQGEISAGDAAVVXTSAQITMGMPYAVTDAVLAPHIGGEAGSSHAPPPALTLIFDRHPIAALLCYPAARYLMGSMTPQAVLAFVALIPPTLPGTNIVLGALPEDRHIDRLAKRQRPGERLDLAMLAAIRRVYGLLANTVRYLQCGGSWREDWGQLSGTAVPPQGAEPQSNAGPRPHIGDTLFTLFRAPELLAPNGDLYNVFAWALDVLAKRLRSMHVFILDYDQSPAGCRDALLQLTSGMVQTHVTTPGSIPTICDLARTFAREMGEAN

SEQ ID NO: 12 - protein, 133 aa, Homo sapiens
MKQLPAATVRLLSSSQIITSVVSVVKELIENSLDAGATSVDVKLENYGFDKIEVRDNGEGIKAVDAPVMAMKYYTSKINSHEDLENLTTYGFRGEALGSICCIAEVLITTRTAADNFSTQYVLDGSGHILSQK

SEQ ID NO: 13 - DNA, 2771 bp, Homo sapiens
cgaggcggatcgggtgttgcatccatggagcgagctgagagctcgagtacagaacctgctaaggccatcaaacctattgatcggaagtcagtccatcagatttgctctgggcaggtggtactgagtctaagcactgcggtaaaggagttagtagaaaacagtctggatgctggtgccactaatattgatctaaagcttaaggactatggagtggatcttattgaagtttcagacaatggatgtggggtagaagaagaaaacttcgaaggcttaactctgaaacatcacacatctaagattcaagagtttgccgacctaactcaggttgaaacttttggctttcggggggaagctctgagctcactttgtgcactgagcgatgtcaccatttctacctgccacgcatcggcgaaggttggaactcgactgatgtttgatcacaatgggaaaattatccagaaaaccccctacccccgccccagagggaccacagtcagcgtgcagcagttattttccacactacctgtgcgccataaggaatttcaaaggaatattaagaaggagtatgccaaaatggtccaggtcttacatgcatactgtatcatttcagcaggcatccgtgtaagttgcaccaatcagcttggacaaggaaaacgacagcctgtggtatgcacaggtggaagccccagcataaaggaaaatatcggctctgtgtttgggcagaagcagttgcaaagcctcattccttttgttcagctgccccctagtgactccgtgtgtgaagagtacggtttgagctgttcggatgctctgcataatcttttttacatctcaggtttcatttcacaatgcacgcatggagttggaaggagttcaacagacagacagtttttctttatcaaccggcggccttgtgacccagcaaaggtctgcagactcgtgaatgaggtctaccacatgtataatcgacaccagtatccatttgttgttcttaacatttctgttgattcagaatgcgttgatatcaatgttactccagataaaaggcaaattttgctacaagaggaaaagcttttgttggcagttttaaagacctctttgataggaatgtttgatagtgatgtcaacaagctaaatgtcagtcagcagccactgctggatgttgaaggtaacttaataaaaatgcatgcagcggatttggaaaagcccatggtagaaaagcaggatcaatccccttcattaaggactggagaagaaaaaaaagacgtgtccatttccagactgcgagaggccttttctcttcgtcacacaacagagaacaagcctcacagcccaaagactccagaaccaagaaggagccctctaggacagaaaaggggtatgctgtcttctagcacttcaggtgccatctctgacaaaggcgtcctgagacctcagaaagaggcagtgagttccagtcacggacccagtgaccctacggacagagcggaggtggagaaggactcggggcacggcagcacttccgtggattctgaggggttcagcatcccagacacgggcagtcactgcagcagcgagtatgcggccagctccccaggggacaggggctcgcaggaacatgtggactctcaggagaaagcgcctgaaactgacgactctttttcagatgtggactgccattcaaaccaggaagataccggatgtaaatttcgagttttgcctcagccaactaatctcgcaaccccaaacacaaagcgttttaaaaaagaagaaattctttccagttctgacatttgtcaaaagttagtaaatactcaggacatgtcagcctctcaggttgatgtagctgtgaaaattaataagaaagttgtgcccctggacttttctatgagttctttagctaaacgaataaagcagttacatcatgaagcacagcaaagtgaaggggaacagaattacaggaagtttagggcaaagatttgtcctggagaaaatcaagcagccgaagatgaactaagaaaagagataagtaaaacgatgtttgcagaaatggaaatcattggtcagtttaacctgggatttataataaccaaactgaatgaggatatcttcatagtggaccagcatgccacggacgagaagtataacttcgagatgctgcagcagcacaccgtgctccaggggcagaggctcatagcacctcagactctcaacttaactgctgttaatgaagctgttctgatagaaaatctggaaatatttagaaagaatggctttgattttgttatcgatgaaaatgctccagtcactgaaagggctaaactgatttccttgccaactagtaaaaactggaccttcggaccccaggacgtcgatgaactgatcttcatgctgagcgacagccctggggtcatgtgccggccttcccgagtcaagcagatgtttgcctccagagcctgccggaagtcggtgatgattgggactgctcttaacacaagcgagatgaagaaactgatcacccacatgggggagatggaccacccctggaactgtccccatggaaggccaaccatgagacacatcgccaacctgggtgtcatttctcagaactgaccgtagtcactgtatggaataattggttttatcgcagatttttatgttttgaaagacagagtcttcactaaccttttttgttttaaaatgaaacctgctacttaaaaaaaatacacatcacacccatttaaaagtgatcttgagaaccttttcaaacc

SEQ ID NO: 14 - DNA, 3063 bp, Homo sapiens
ggcacgagtggctgcttgcggctagtggatggtaattgcctgcctcgcgctagcagcaagctgctctgttaaaagcgaaaatgaaacaattgcctgcggcaacagttcgactcctttcaagttctcagatcatcacttcggtggtcagtgttgtaaaagagcttattgaaaactccttggatgctggtgccacaagcgtagatgttaaactggagaactatggatttgataaaattgaggtgcgagataacggggagggtatcaaggctgttgatgcacctgtaatggcaatgaagtactacacctcaaaaataaatagtcatgaagatcttgaaaatttgacaacttacggttttcgtggagaagccttggggtcaatttgttgtatagctgaggttttaattacaacaagaacggctgctgataattttagcacccagtatgttttagatggcagtggccacatactttctcagaaaccttcacatcttggtcaaggtacaactgtaactgctttaagattatttaagaatctacctgtaagaaagcagttttactcaactgcaaaaaaatgtaaagatgaaataaaaaagatccaagatctcctcatgagctttggtatccttaaacctgacttaaggattgtctttgtacataacaaggcagttatttggcagaaaagcagagtatcagatcacaagatggctctcatgtcagttctggggactgctgttatgaacaatatggaatcctttcagtaccactctgaagaatctcagatttatctcagtggatttcttccaaagtgtgatgcagaccactctttcactagtctttcaacaccagaaagaagtttcatcttcataaacagtcgaccagtacatcaaaaagatatcttaaagttaatccgacatcattacaatctgaaatgcctaaaggaatctactcgtttgtatcctgttttctttctgaaaatcgatgttcctacagctgatgttgatgtaaatttaacaccagataaaagccaagtattattacaaaataaggaatctgttttaattgctcttgaaaatctgatgacgacttgttatggaccattacctagtacaaattcttatgaaaataataaaacagatgtttccgcagctgacatcgttcttagtaaaacagcagaaacagatgtgctttttaataaagtggaatcatctggaaagaattattcaaatgttgatacttcagtcattccattccaaaatgatatgcataatgatgaatctggaaaaaacactgatgattgtttaaatcaccagataagtattggtgactttggttatggtcattgtagtagtgaaatttctaacattgataaaaacactaagaatgcatttcaggacatttcaatgagtaatgtatcatgggagaactctcagacggaatatagtaaaacttgttttataagttccgttaagcacacccagtcagaaaatggcaataaagaccatatagatgagagtggggaaaatgaggaagaagcaggtcttgaaaactcttcggaaatttctgcagatgagtggagcaggggaaatatacttaaaaattcagtgggagagaatattgaacctgtgaaaattttagtgcctgaaaaaagtttaccatgtaaagtaagtaataataattatccaatccctgaacaaatgaatcttaatgaagattcatgtaacaaaaaatcaaatgtaatagataataaatctggaaaagttacagcttatgatttacttagcaatcgagtaatcaagaaacccatgtcagcaagtgctctttttgttcaagatcatcgtcctcagtttctcatagaaaatcctaagactagtttagaggatgcaacactacaaattgaagaactgtggaagacattgagtgaagaggaaaaactgaaatatgaagagaaggctactaaagacttggaacgatacaatagtcaaatgaagagagccattgaacaggagtcacaaatgtcactaaaagatggcagaaaaaagataaaacccaccagcgcatggaatttggcccagaagcacaagttaaaaacctcattatctaatcaaccaaaacttgatgaactccttcagtcccaaattgaaaaaagaaggagtcaaaatattaaaatggtacagatccccttttctatgaaaaacttaaaaataaattttaagaaacaaaacaaagttgacttagaagagaaggatgaaccttgcttgatccacaatctcaggtttcctgatgcatggctaatgacatccaaaacagaggtaatgttattaaatccatatagagtagaagaagccctgctatttaaaagacttcttgagaatcataaacttcctgcagagccactggaaaagccaattatgttaacagagagtctttttaatggatctcattatttagacgttttatataaaatgacagcagatgaccaaagatacagtggatcaacttacctgtctgatcctcgtcttacagcgaatggtttcaagataaaattgataccaggagtttcaattactgaaaattacttggaaatagaaggaatggctaattgtctcccattctatggagtagcagatttaaaagaaattcttaatgctatattaaacagaaatgcaaaggaagtttatgaatgtagacctcgcaaagtgataagttatttagagggagaagcagtgcgtctatccagacaattacccatgtacttatcaaaagaggacatccaagacattatctacagaatgaagcaccagtttggaaatgaaattaaagagtgtgttcatggtcgcccattttttcatcatttaacctatcttccagaaactacatgattaaatatgtttaagaagattagttaccattgaaattggttctgtcataaaacagcatgagtctggttttaaattatctttgtattatgtgtcacatggttattttttaaatgaggattcactgacttgtttttatattgaaaaaagttccacgtattgtagaaaacgtaaataaactaataac

SEQ ID NO: 15 - DNA, 3145 bp, Homo sapiens
ggcgggaaacagcttagtgggtgtggggtcgcgcattttcttcaaccaggaggtgaggaggtttcgacatggcggtgcagccgaaggagacgctgcagttggagagcgcggccgaggtcggcttcgtgcgcttctttcagggcatgccggagaagccgaccaccacagtgcgccttttcgaccggggcgacttctatacggcgcacggcgaggacgcgctgctggccgcccgggaggtgttcaagacccagggggtgatcaagtacatggggccggcaggagcaaagaatctgcagagtgttgtgcttagtaaaatgaattttgaatcttttgtaaaagatcttcttctggttcgtcagtatagagttgaagtttataagaatagagctggaaataaggcatccaaggagaatgattggtatttggcatataaggcttctcctggcaatctctctcagtttgaagacattctctttggtaacaatgatatgtcagcttccattggtgttgtgggtgttaaaatgtccgcagttgatggccagagacaggttggagttgggtatgtggattccatacagaggaaactaggactgtgtgaattccctgataatgatcagttctccaatcttgaggctctcctcatccagattggaccaaaggaatgtgttttacccggaggagagactgctggagacatggggaaactgagacagataattcaaagaggaggaattctgatcacagaaagaaaaaaagctgacttttccacaaaagacatttatcaggacctcaaccggttgttgaaaggcaaaaagggagagcagatgaatagtgctgtattgccagaaatggagaatcaggttgcagtttcatcactgtctgcggtaatcaagtttttagaactcttatcagatgattccaactttggacagtttgaactgactacttttgacttcagccagtatatgaaattggatattgcagcagtcagagcccttaacctttttcagggttctgttgaagataccactggctctcagtctctggctgccttgctgaataagtgtaaaacccctcaaggacaaagacttgttaaccagtggattaagcagcctctcatggataagaacagaatagaggagagattgaatttagtggaagcttttgtagaagatgcagaattgaggcagactttacaagaagatttacttcgtcgattcccagatcttaaccgacttgccaagaagtttcaaagacaagcagcaaacttacaagattgttaccgactctatcagggtataaatcaactacctaatgttatacaggctctggaaaaacatgaaggaaaacaccagaaattattgttggcagtttttgtgactcctcttactgatcttcgttctgacttctccaagtttcaggaaatgatagaaacaactttagatatggatcaggtggaaaaccatgaattccttgtaaaaccttcatttgatcctaatctcagtgaattaagagaaataatgaatgacttggaaaagaagatgcagtcaacattaataagtgcagccagagatcttggcttggaccctggcaaacagattaaactggattccagtgcacagtttggatattactttcgtgtaacctgtaaggaagaaaaagtccttcgtaacaataaaaactttagtactgtagatatccagaagaatggtgttaaatttaccaacagcaaattgacttctttaaatgaagagtataccaaaaataaaacagaatatgaagaagcccaggatgccattgttaaagaaattgtcaatatttcttcaggctatgtagaaccaatgcagacactcaatgatgtgttagctcagctagatgctgttgtcagctttgctcacgtgtcaaatggagcacctgttccatatgtacgaccagccattttggagaaaggacaaggaagaattatattaaaagcatccaggcatgcttgtgttgaagttcaagatgaaattgcatttattcctaatgacgtatactttgaaaaagataaacagatgttccacatcattactggccccaatatgggaggtaaatcaacatatattcgacaaactggggtgatagtactcatggcccaaattgggtgttttgtgccatgtgagtcagcagaagtgtccattgtggactgcatcttagcccgagtaggggctggtgacagtcaattgaaaggagtctccacgttcatggctgaaatgttggaaactgcttctatcctcaggtctgcaaccaaagattcattaataatcatagatgaattgggaagaggaacttctacctacgatggatttgggttagcatgggctatatcagaatacattgcaacaaagattggtgctttttgcatgtttgcaacccattttcatgaacttactgccttggccaatcagataccaactgttaataatctacatgtcacagcactcaccactgaagagaccttaactatgctttatcaggtgaagaaaggtgtctgtgatcaaagttttgggattcatgttgcagagcttgctaatttccctaagcatgtaatagagtgtgctaaacagaaagccctggaacttgaggagtttcagtatattggagaatcgcaaggatatgatatcatggaaccagcagcaaagaagtgctatctggaaagagagcaaggtgaaaaaattattcaggagttcctgtccaaggtgaaacaaatgccctttactgaaatgtcagaagaaaacatcacaataaagttaaaacagctaaaagctgaagtaatagcaaagaataatagctttgtaaatgaaatcatttcacgaataaaagttactacgtgaaaaatcccagtaatggaatgaaggtaatattgataagctattgtctgtaatagttttatattgttttatattaaccctttttccatagtgttaactgtcagtgcccatgggctatcaacttaataagatatttagtaatattttactttgaggacattttcaaagatttttattttgaaaaatgagagctgtaactgaggactgtttgcaattgacataggcaataataagtgatgtgctgaattttataaataaaatcatgtagtttgtgg

SEQ ID NO: 16 - DNA, 2484 bp, Homo sapiens
cttggctcttctggcgccaaaatgtcgttcgtggcaggggttattcggcggctggacgagacagtggtgaaccgcatcgcggcgggggaagttatccagcggccagctaatgctatcaaagagatgattgagaactgtttagatgcaaaatccacaagtattcaagtgattgttaaagagggaggcctgaagttgattcagatccaagacaatggcaccgggatcaggaaagaagatctggatattgtatgtgaaaggttcactactagtaaactgcagtcctttgaggatttagccagtatttctacctatggctttcgaggtgaggctttggccagcataagccatgtggctcatgttactattacaacgaaaacagctgatggaaagtgtgcatacagagcaagttactcagatggaaaactgaaagcccctcctaaaccatgtgctggcaatcaagggacccagatcacggtggaggaccttttttacaacatagccacgaggagaaaagctttaaaaaatccaagtgaagaatatgggaaaattttggaagttgttggcaggtattcagtacacaatgcaggcattagtttctcagttaaaaaacaaggagagacagtagctgatgttaggacactacccaatgcctcaaccgtggacaatattcgctccatctttggaaatgctgttagtcgagaactgatagaaattggatgtgaggataaaaccctagccttcaaaatgaatggttacatatccaatgcaaactactcagtgaagaagtgcatcttcttactcttcatcaaccatcgtctggtagaatcaacttccttgagaaaagccatagaaacagtgtatgcagcctatttgcccaaaaacacacacccattcctgtacctcagtttagaaatcagtccccagaatgtggatgttaatgtgcaccccacaaagcatgaagttcacttcctgcacgaggagagcatcctggagcgggtgcagcagcacatcgagagcaagctcctgggctccaattcctccaggatgtacttcacccagactttgctaccaggacttgctggcccctctggggagatggttaaatccacaacaagtctgacctcgtcttctacttctggaagtagtgataaggtctatgcccaccagatggttcgtacagattcccgggaacagaagcttgatgcatttctgcagcctctgagcaaacccctgtccagtcagccccaggccattgtcacagaggataagacagatatttctagtggcagggctaggcagcaagatgaggagatgcttgaactcccagcccctgctgaagtggctgccaaaaatcagagcttggagggggatacaacaaaggggacttcagaaatgtcagagaagagaggacctacttccagcaaccccagaaagagacatcgggaagattctgatgtggaaatggtggaagatgattcccgaaaggaaatgactgcagcttgtaccccccggagaaggatcattaacctcactagtgttttgagtctccaggaagaaattaatgagcagggacatgaggttctccgggagatgttgcataaccactccttcgtgggctgtgtgaatcctcagtgggccttggcacagcatcaaaccaagttataccttctcaacaccaccaagcttagtgaagaactgttctaccagatactcatttatgattttgccaattttggtgttctcaggttatcggagccagcaccgctctttgaccttgccatgcttgccttagatagtccagagagtggctggacagaggaagatggtcccaaagaaggacttgctgaatacattgttgagtttctgaagaagaaggctgagatgcttgcagactatttctctttggaaattgatgaggaagggaacctgattggattaccccttctgattgacaactatgtgccccctttggagggactgcctatcttcattcttcgactagccactgaggtgaattgggacgaagaaaaggaatgttttgaaagcctcagtaaagaatgcgctatgttctattccatccggaagcagtacatatctgaggagtcgaccctctcaggccagcagagtgaagtgcctggctccattccaaactcctggaagtggactgtggaacacattgtctataaagccttgcgctcacacattctgcctcctaaacatttcacagaagatggaaatatcctgcagcttgctaacctgcctgatctatacaaagtctttgagaggtgttaaatatggttatttatgcactgtgggatgtgttcttctttctctgtattccgatacaaagtgttgtatcaaagtgtgatatacaaagtgtaccaacataagtgttggtagcacttaagacttatacttgccttctgatagtattcctttatacacagtggattgattataaataaatagatgtgtcttaacata

SEQ ID NO: 17 - DNA, 1408 bp, Homo sapiens
ggcgctcctacctgcaagtggctagtgccaagtgctgggccgccgctcctgccgtgcatgttggggagccagtacatgcaggtgggctccacacggagaggggcgcagacccggtgacagggctttacctggtacatcggcatggcgcaaccaaagcaagagagggtggcgcgtgccagacaccaacggtcggaaaccgccagacaccaacggtcggaaaccgccaagacaccaacgctcggaaaccgccagacaccaacgctcggaaaccgccagacaccaaggctcggaatccacgccaggccacgacggagggcgactacctcccttctgaccctgctgctggcgttcggaaaaaacgcagtccggtgtgctctgattggtccaggctctttgacgtcacggactcgacctttgacagagccactaggcgaaaaggagagacgggaagtattttttccgccccgcccggaaagggtggagcacaacgtcgaaagcagccgttgggagcccaggaggcggggcgcctgtgggagccgtggagggaactttcccagtccccgaggcggatccggtgttgcatccttggagcgagctgagaactcgagtacagaacctgctaaggccatcaaacctattgatcggaagtcagtccatcagatttgctctgggccggtggtaccgagtctaaggccgaatgcggtgaaggagttagtagaaaacagtctggatgctggtgccactaatgttgatctaaagcttaaggactatggagtggatctcattgaagtttcaggcaatggatgtggggtagaagaagaaaacttcgaaggctttactctgaaacatcacacatgtaagattcaagagtttgccgacctaactcaggtggaaacttttggctttcggggggaagctctgagctcactttgtgcactgagtgatgtcaccatttctacctgccgtgtatcagcgaaggttgggactcgactggtgtttgatcactatgggaaaatcatccagaaaaccccctacccccgccccagagggatgacagtcagcgtgaagcagttattttctacgctacctgtgcaccataaagaatttcaaaggaatattaagaagaaacgtgcctgcttccccttcgccttctgccgtgattgtcagtttcctgaggcctccccagccatgcttcctgtacagcctgtagaactgactcctagaagtaccccaccccacccctgctccttggaggacaacgtgatcactgtattcagctctgtcaagaatggtccaggttcttctagatgatctgcacaaatggttcctctcctccttcctgatgtctgccattagcattggaataaagttcctgctgaaaatccaaaaaaaaaaaaaaaaaaaaaaa

SEQ ID NO: 18 - protein, 389 aa, Homo sapiens
MAQPKQERVARARHQRSETARHQRSETAKTPTLGNRQTPTLGNRQTPRLGIHARPRRRATTSLLTLLLAFGKNAVRCALIGPGSLTSRTRPLTEPLGEKERREVFFPPRPERVEHNVESSRWEPRRRGACGSRGGNFPSPRGGSGVASLERAENSSTEPAKAIKPIDRKSVHQICSGPVVPSLRPNAVKELVENSLDAGATNVDLKLKDYGVDLIEVSGNGCGVEEENFEGFTLKHHTCKIQEFADLTQVETFGFRGEALSSLCALSDVTISTCRVSAKVGTRLVFDHYGKIIQKTPYPRPRGMTVSVKQLFSTLPVHHKEFQRNIKKKRACFPFAFCRDCQFPEASPAMLPVQPVELTPRSTPPHPCSLEDNVITVFSSVKNGPGSSR

SEQ ID NO: 19 - DNA, 1785 bp, Homo sapiens
tttttagaaactgatgtttattttccatcaaccatttttccatgctgcttaagagaatatgcaagaacagcttaagaccagtcagtggttgctcctacccattcagtggcctgagcagtggggagctgcagaccagtcttccgtggcaggctgagcgctccagtcttcagtagggaattgctgaataggcacagagggcacctgtacaccttcagaccagtctgcaacctcaggctgagtagcagtgaactcaggagcgggagcagtccattcaccctgaaattcctccttggtcactgccttctcagcagcagcctgctcttctttttcaatctcttcaggatctctgtagaagtacagatcaggcatgacctcccatgggtgttcacgggaaatggtgccacgcatgcgcagaacttcccgagccagcatccaccacattaaacccactgagtgagctcccttgttgttgcatgggatggcaatgtccacatagcgcagaggagaatctgtgttacacagcgcaatggtaggtaggttaacataagatgcctccgtgagaggcgaaggggcggcgggacccgggcctggcccgtatgtgtccttggcggcctagactaggccgtcgctgtatggtgagccccagggaggcggatctgggcccccagaaggacacccgcctggatttgccccgtagcccggcccgggcccctcgggagcagaacagccttggtgaggtggacaggaggggacctcgcgagcagacgcgcgcgccagcgacagcagccccgccccggcctctcgggagccggggggcagaggctgcggagccccaggagggtctatcagccacagtctctgcatgtttccaagagcaacaggaaatgaacacattgcaggggccagtgtcattcaaagatgtggctgtggatttcacccaggaggagtggcggcaactggaccctgatgagaagatagcatacggggatgtgatgttggagaactacagccatctagtttctgtggggtatgattatcaccaagccaaacatcatcatggagtggaggtgaaggaagtggagcagggagaggagccgtggataatggaaggtgaatttccatgtcaacatagtccagaacctgctaaggccatcaaacctattgatcggaagtcagtccatcagatttgctctgggccagtggtactgagtctaagcactgcagtgaaggagttagtagaaaacagtctggatgctggtgccactaatattgatctaaagcttaaggactatggagtggatctcattgaagtttcagacaatggatgtggggtagaagaagaaaactttgaaggcttaatctctttcagctctgaaacatcacacatgtaagattcaagagtttgccgacctaactgaagttgaaactttcggttttcagggggaagctctgagctcactgtgtgcactgagcgatgtcaccatttctacctgccacgcgttggtgaaggttgggactcgactggtgtttgatcacgatgggaaaatcatccaggaaaccccctacccccaccccagagggaccacagtcagcgtgaagcagttattttctacgctacctgtgcgccataaggaatttcaaaggaatattaagaagacgtgcctgcttccccttcgccttctgccgtgattgtcagtttcctgaggcctccccagccatgcttcctgtacagcctgcagaactgtgagtcaattaaacctcttttcttcataaattaaaaaaaaa

SEQ ID NO: 20 - protein, 264 aa, Homo sapiens
MCPWRPRLGRRCMVSPREADLGPQKDTRLDLPRSPARAPREQNSLGEVDRRGPREQTRAPATAAPPRPLGSRGAEAAEPQEGLSATVSACFQEQQEMNTLQGPVSFKDVAVDFTQEEWRQLDPDEKIAYGDVMLENYSHLVSVGYDYHQAKHHHGVEVKEVEQGEEPWIMEGEFPCQHSPEPAKAIKPIDRKSVHQICSGPVVLSLSTAVKELVENSLDAGATNIDLKLKDYGVDLIEVSDNGCGVEEENFEGLISFSSETSHM

SEQ ID NO: 21 - protein, 1453 aa, Homo sapiens
MIKCLSVEVQAKLRSGLAISSLGQCVEELALNSIDAEAKCVAVRVNMETFQVQVIDNGFGMGSDDVEKVGNRYFTSKCHSVQDLENPRFYGFRGEALANIADMASAVEISSKKNRTMKTFVKLFQSGKALKACEADVTRASAGTTVTVYNLFYQLPVRRKCMDPRLEFEKVRQRIEALSLMHPSISFSLRNDVSGSMVLQLPKTKDVCSRFCQIYGLGKSQKLREISFKYKEFELSGYISSEAHYNKNMQFLFVNKRLVLRTKLHKLIDFLLRKESIICKPKNGPTSRQMNSSLRHRSTPELYGIYVINVQCQFCEYDVCMEPAKTLIEFQNWDTLLFCIQEGVKMFLKQEKLFVELSGEDIKEFSEDNGFSLFDATLQKRVTSDERSNFQEACNNILDSYEMFNLQSKAVKRKTTAENVNTQSSRDSEATRKNTNDAFLYIYESGGPGHSKMTEPSLQNKDSSCSESKMLEQETIVASEAGENEKHKKSFLEHSSLENPCGTSLEMFLSPFQTPCHFEESGQDLEIWKESTTVNGMAANILKNNRIQNQPKRFKDATEVGCQPLPFATTLWGVHSAQTEKEKKKESSNCGRRNVFSYGRVKLCSTGFITHVVQNEKTKSTETEHSFKNYVRPGPTRAQETFGNRTRHSVETPDIKDLASTLSKESGQLPNKKNCRTNISYGLENEPTATYTMFSAFQEGSKKSQTDCILSDTSPSFPWYRHVSNDSRKTDKLIGFSKPIVRKKLSLSSQLGSLEKFKRQYGKVENPLDTEVEESNGVTTNLSLQVEPDILLKDKNRLENSDVCKITTMEHSDSDSSCQPASHILDSEKFPFSKDEDCLEQQMPSLRESPMTLKELSLFNRKPLDLEKSSESLASKLSRLKGSERETQTMGMMSRFNELPNSDSSRKDSKLCSVLTQDFCMLFNNKHEKTENGVIPTSDSATQDNSFNKNSKTHSNSNTTENCVISETPLVLPYNNSKVTGKDSDVLIRASEQQIGSLDSPSGMLMNPVEDATGDQNGICFQSEESKARACSETEESNTCCSDWQRHFDVALGRMVYVNKMTGLSTFIAPTEDIQAACTKDLTTVAVDVVLENGSQYRCQPFRSDLVLPFLPRARAERTVMRQDNRDTVDDTVSSESLQSLFSEWDNPVFARYPEVAVDVSSGQAESLAVKIHNILYPYRFTKGMIHSMQVLQQVDNKFIACLMSTKTEENGEAGGNLLVLVDQHAAHERIRLEQLIIDSYEKQQAQGSGRKKLLSSTLIPPLEITVTEEQRRLLWCYHKNLEDLGLEFVFPDTSDSLVLVGKVPLCFVEREANELRRGRSTVTKSIVEEFIREQLELLQTTGGIQGTLPLTVQKVLASQACHGAIKFNDGLSLQESCRLIEALSSCQLPFQCAHGRPSMLPLADIDHLEQEKQIKPNLTKLRKMAQAWRLFGKAECDTRQSLQQSMPPCEPP

SEQ ID NO: 22 - DNA, 4895 bp, Homo sapiens
gtcggcgtccgaggcggttggtgtcggagaatttgttaagcgggactccaggcaattatttccagtcagagaaggaaaccagtgcctggcattctcaccatctttctacctaccatgatcaagtgcttgtcagttgaagtacaagccaaattgcgttctggtttggccataagctccttgggccaatgtgttgaggaacttgccctcaacagtattgatgctgaagcaaaatgtgtggctgtcagggtgaatatggaaaccttccaagttcaagtgatagacaatggatttgggatggggagtgatgatgtagagaaagtgggaaatcgttatttcaccagtaaatgccactcggtacaggacttggagaatccaaggttttatggtttccgaggagaggccttggcaaatattgctgacatggccagtgctgtggaaatttcgtccaagaaaaacaggacaatgaaaacttttgtgaaactgtttcagagtggaaaagccctgaaagcttgtgaagctgatgtgactagagcaagcgctgggactactgtaacagtgtataacctattttaccagcttcctgtaaggaggaaatgcatggaccctagactggagtttgagaaggttaggcagagaatagaagctctctcactcatgcacccttccatttctttctctttgagaaatgatgtttctggttccatggttcttcagctccctaaaaccaaagacgtatgttcccgattttgtcaaatttatggattgggaaagtcccaaaagctaagagaaataagttttaaatataaagagtttgagcttagtggctatatcagctctgaagcacattacaacaagaatatgcagtttttgtttgtgaacaaaagactagttttaaggacaaagctacataaactcattgactttttattaaggaaagaaagtattatatgcaagccaaagaatggtcccaccagtaggcaaatgaattcaagtcttcggcaccggtctaccccagaactctatggcatatatgtaattaatgtgcagtgccaattctgtgagtatgatgtgtgcatggagccagccaaaactctgattgaatttcagaactgggacactctcttgttttgcattcaggaaggagtgaaaatgtttttaaagcaagaaaaattatttgtggaattatcaggtgaggatattaaggaatttagtgaagataatggttttagtttatttgatgctactcttcagaagcgtgtgacttccgatgagaggagcaatttccaggaagcatgtaataatattttagattcctatgagatgtttaatttgcagtcaaaagctgtgaaaagaaaaactactgcagaaaacgtaaacacacagagttctagggattcagaagctaccagaaaaaatacaaatgatgcatttttgtacatttatgaatcaggtggtccaggccatagcaaaatgacagagccatctttacaaaacaaagacagctcttgctcagaatcaaagatgttagaacaagagacaattgtagcatcagaagctggtgaaaatgagaaacataaaaaatctttcctggaacgtagctctttagaaaatccgtgtggaaccagtttagaaatgtttttaagcccttttcagacaccatgtcactttgaggagagtgggcaggatctagaaatatggaaagaaagtactactgttaatggcatggctgccaacatcttgaaaaataatagaattcagaatcaaccaaagagatttaaagatgctactgaagtgggatgccagcctctgccttttgcaacaacattatggggagtacatagtgctcagacagagaaagagaaaaaaaaagaatctagcaattgtggaagaagaaatgtttttagttatgggcgagttaaattatgttccactggctttataactcatgtagtacaaaatgaaaaaactaaatcaactgaaacagaacattcatttaaaaattatgttagacctggtcccacacgtgcccaagaaacatttggaaatagaacacgtcattcagttgaaactccagacatcaaagatttagccagcactttaagtaaagaatctggtcaattgcccaacaaaaaaaattgcagaacgaatataagttatgggctagagaatgaacctacagcaacttatacaatgttttctgcttttcaggaaggtagcaaaaaatcacaaacagattgcatattatctgatacatccccctctttcccctggtatagacacgtttccaatgatagtaggaaaacagataaattaattggtttctccaaaccaatcgtccgtaagaagctaagcttgagttcacagctaggatctttagagaagtttaagaggcaatatgggaaggttgaaaatcctctggatacagaagtagaggaaagtaatggagtcactaccaatctcagtcttcaagttgaacctgacattctgctgaaggacaagaaccgcttagagaactctgatgtttgtaaaatcactactatggagcatagtgattcagatagtagttgtcaaccagcaagccacatccttgactcagagaagtttccattctccaaggatgaagattgtttagaacaacagatgcctagtttgagagaaagtcctatgaccctgaaggagttatctctctttaatagaaaacctttggaccttgagaagtcatctgaatcactagcctctaaattatccagactgaagggttccgaaagagaaactcaaacaatggggatgatgagtcgttttaatgaacttccaaattcagattccagtaggaaagacagcaagttgtgcagtgtgttaacacaagatttttgtatgttatttaacaacaagcatgaaaaaacagagaatggtgtcatcccaacatcagattctgccacacaggataattcctttaataaaaatagtaaaacacattctaacagcaatacaacagagaactgtgtgatatcagaaactcctttggtattgccctataataattctaaagttaccggtaaagattcagatgttcttatcagagcctcagaacaacagataggaagtcttgactctcccagtggaatgttaatgaatccggtagaagatgccacaggtgaccaaaatggaatttgttttcagagtgaggaatctaaagcaagagcttgttctgaaactgaagagtcaaacacgtgttgttcagattggcagcggcatttcgatgtagccctgggaagaatggtttatgtcaacaaaatgactggactcagcacattcattgccccaactgaggacattcaggctgcttgtactaaagacctgacaactgtggctgtggatgttgtacttgagaatgggtctcagtacaggtgtcaaccttttagaagcgaccttgttcttcctttccttccgagagctcgagcagagaggactgtgatgagacaggataacagagatactgtggatgatactgttagtagcgaatcgcttcagtctttgttctcagaatgggacaatccagtatttgcccgttatccagaggttgctgttgatgtaagcagtggccaggctgagagcttagcagttaaaattcacaacatcttgtatccctatcgtttcaccaaaggaatgattcattcaatgcaggttctccagcaagtagataacaagtttattgcctgtttgatgagcactaagactgaagagaatggcgaggcagattcctacgagaagcaacaggcacaaggctctggtcggaaaaaattactgtcttctactctaattcctccgctagagataacagtgacagaggaacaaaggagactcttatggtgttaccacaaaaatctggaagatctgggccttgaatttgtatttccagacactagtgattctctggtccttgtgggaaaagtaccactatgttttgtggaaagagaagccaatgaacttcggagaggaagatctactgtgaccaagagtattgtggaggaatttatccgagaacaactggagctactccagaccaccggaggcatccaagggacattgccactgactgtccagaaggtgttggcatcccaagcctgccatggggccattaagtttaatgatggcctgagcttacaggaaagttgccgccttattgaagctctgtcctcatgccagctgccattccagtgtgctcacgggagaccttctatgctgccgttagctgacatagaccacttggaacaggaaaaacagattaaacccaacctcactaaacttcgcaaaatggcccaggcctggcgtctctttggaaaagcagagtgtgatacaaggcagagcctgcagcagtccatgcctccctgtgagccaccatgagaacagaatcactggtctaaaaggaacaaagggatgttcactgtatgcctctgagcagagagcagcagcagcaggtaccagcacggccctgactgaatcagcccagtgtccctgagcagcttagacagcagggctctctgtatcagtctttcttgagcagatgattcccctagttgagtagccagatgaaattcaagcctaaagacaattcattcatttgcatccatgggcacagaaggttgctatatagtatctaccttttgctacttatttaatgataaaatttaatgacagtttaaaaaaaaaaaaaaaaaaaattatttgaaggggtgggtgatttttgtttttgtacagttttttttcaagcttcacatttgcgtgtatctaattcagctgatgctcaagtccaaggggtagtctgccttcccaggctgcccccagggtttctgcactggtcccctcttttcccttcagtcttcttcacttccctt

SEQ ID NO: 23 - protein, 16 aa, Homo sapiens
AVKELVENSLDAGATN

SEQ ID NO: 24 - protein, 48 aa, Homo sapiens
LRPNAVKELVENSLDAGATNVDLKLKDYGVDLIEVSGNGCGVEEENFE

SEQ ID NO: 25 - DNA, 21 bp, Homo sapiens
tgactacttttgacttcagcc

SEQ ID NO: 26 - DNA, 22 bp, Homo sapiens
aaccattcaacatttttaaccc